(19) United States
(12) Patent Application Publication
(10) Pub. No.: US 2012/0164242 A1
(43) Pub. Date: Jun. 28, 2012

(54) NOVEL METHODS FOR MODULATING MELANIN PRODUCTION

(76) Inventors: Wen-Jen Yu, Taichung (TW); Sue-Joan Chang, Tainan City (TW); Wei-Chi Tseng, Taichung City (TW)

(21) Appl. No.: 12/979,218

(22) Filed: Dec. 27, 2010

(51) Int. Cl. A61K 36/704; A61P 17/00; A61K 31/7034

(52) U.S. Cl. 424/725; 514/25

(57) ABSTRACT

Methods are provided for modulating pigmentation and melanin production by administering an extract of Polygonum multiflorum Thunb (PMT). Methods are also provided for treating conditions that may be regulated or associated with abnormal melanogenesis.

### TECHNICAL FIELD

The present application relates to novel methods of using Polygonum multiflorum Thunb (PMT) for modulating pigmentation and melanin production as well as conditions that may be regulated or associated with melanogenesis.

### BACKGROUND

There is a substantial desire shared by many people to darken their gray hair. Most hair coloring agents rely on harsh chemicals such as hydrogen peroxide or ammonia to strip the hair and allow it to absorb the chemical color being applied to it. They may weaken and destroy hair shaft over time. Additionally, many of chemical hair colorants use various ingredients that have been found, over time, to increase the long-term risk of various forms of cancer or other diseases (Robbins, Clarence. Chemical and Physical Behavior of Human Hair. New York: Springer-Verlag, 2002, 342-343). On the other hand, non-chemical methods which may induce oxidation of hair proteins including exposure to the sun and/or to the rays of ultraviolet lights, as in the case of skin tanning procedures, may be harmful and are time consuming (Robbins, Clarence. Chemical and Physical Behavior of Human Hair. New York: Springer-Verlag, 2002, 163-165).

### BRIEF SUMMARY

The present application relates to the discovery of novel methods for modulating pigmentation and melanin production using an extract of Polygonum multiflorum Thunb (PMT). Accordingly, the present application describes novel methods of using an extract of PMT for modulating pigmentation and melanin production as well as treating diseases and conditions that may be associated with melanin deficiency.

In one embodiment, the present application relates to a method for modulating pigmentation of hair, skin, nail and/or eyelashes comprising administering to a subject in need thereof an effective amount of an extract of Polygonum multiflorum Thunb (PMT).

In another embodiment, the present application relates to a method for modulating melanin production comprising administering to a subject in need thereof an effective amount of an extract of Polygonum multiflorum Thunb (PMT).

In another embodiment, the present application relates to a method for treating a disease which exhibits a melanin deficiency comprising administering to a subject in need thereof an effective amount of an extract of Polygonum multiflorum Thunb (PMT).

In some embodiments, the modulation increases pigmentation of the hair, skin, nail and/or eyelashes.

In other embodiments, the modulation increases melanin production.

In some embodiments, the extract of Polygonum multiflorum Thunb (PMT) is effective in increasing tyrosinase expression and/or activity.

In some embodiments, the extract of Polygonum multiflorum Thunb (PMT) is effective in increasing tyrosinase-related protein 2 expression and/or activity.

In some embodiments, the subject is a human.

In some embodiments, the extract of PMT is administered orally, topically, intravenously, intraperitoneally, subcutaneously, intramuscularly, intrathecally, intradermally, nasally, enterically, pessaries, suppositories. In some embodiments, the extract of PMT is administered orally or topically.

In some embodiments, the extract of PMT comprises 2,3,5,4′-tetrahydroxystilbene-2-O-beta-D-glucoside (THSG).

This Brief Summary is provided to introduce simplified concepts related to methods of using Polygonum multiflorum Thunb (PMT), which are further described below in the Detailed Description. This summary is not intended to identify essential features of the claimed subject matter, nor should it be used to limit the scope of the claims.

### BRIEF DESCRIPTION OF THE DRAWINGS

FIG. 1 is an exemplary flow chart illustrating the method of extracting the root of PMT according to the present application.

FIG. 2 is a graph showing the melanin content in B16-F10 cells following treatment with different concentrations of PMT extract (1, 5, 10 and 20 μg/mL). “Control” represents cells treated with control medium (Dulbecco's modified Eagle's medium (DMEM)); “0.1% EtOH” represents cells treated with vehicle medium (DMEM containing 0.1% ethanol (EtOH)). The melanin content in B16-F10 cells following treatment with PMT extract (10 μg/mL) is significantly higher than untreated cells. p<0.05. (*).

FIG. 3 is a graph showing B16-F10 cell survival rates in response to different concentrations of PMT extract (1, 5, 10 and 20 μg/mL). Cells were treated with PMT extract for 48 hours and cell survival was assessed. “C” represents cells treated with control medium (Dulbecco's modified Eagle's medium (DMEM)); “V” or “Vehicle” represents cells treated with vehicle medium (DMEM containing 0.1% ethanol (EtOH)).

FIG. 4 is a series of photographs showing morphological changes of B16-F10 cells in response to different concentrations of PMT extract (1, 5, 10 and 20 μg/mL) treated for 48 hours. “C” represents cells treated with control medium (DMEM); “0.1% EtOH” represents cells treated with vehicle medium (DMEM containing 0.1% ethanol (EtOH)).

FIG. 5 is a photograph of an agarose gel electrophoresis showing mRNA level changes of melanin related genes in response to PMT extract. B16-F10 cells were treated with different concentrations of PMT extract (1, 5, 10 and 20 μg/mL) for 48 hours. RT-PCR was performed on these cells and mRNA levels of Tyrosinase and its related proteins (TRP1, TRP2, p53 and p21) were evaluated. The mRNA levels of the housekeeping GAPDH was also measured as a control. “C” represents cells treated with control medium (DMEM); “−” represents the negative control.

FIG. 6 is a photograph of a Western blot analysis of tyrosinase expression (normalized to β-actin expression) in B16-F10 cells in response to PMT extract. B16-F10 cells were treated with different concentrations of PMT extract (1, 5, 10 and 20 μg/mL) for 48 hours. Proteins were separated by SDS-PAGE and were further analyzed by Western blot. Tyrosinase was identified by an antibody against tyrosinase. “C” represents cells treated with control medium (DMEM); “V” represents cells treated with vehicle medium (DMEM containing 0.1% ethanol).

FIG. 7 is a photograph of a Western blot analysis of TRP-2 (tyrosinase-related protein 2) expression (normalized to β-actin expression) in B16-F10 cells in response to PMT extract. B16-F10 cells were treated with different concentrations of PMT extract (1, 5, 10 and 20 μg/mL) for 48 hours. Proteins were separated by SDS-PAGE and were further analyzed by Western blot. TRP-2 was identified by an antibody against TRP-2. “C” represents cells treated with control medium (DMEM); “V” represents cells treated with vehicle medium (DMEM containing 0.1% ethanol).

FIG. 8A is a photograph showing the comparison of the color density of newly grown hairs between mice given with regular water (RO water) and mice given PMT extract (1 g/kg) by oral gavage. The mice were anesthetized by 50 mg/mL pentobarbital and aligned for photograph. The region of interest (ROl) of newly formed hairs was selected by free hand function of MultiGauge V3.0 (Fujifilm) and the QL (Quantum Level) values of the ROl were then measured. The QL/Pixel² represents the color density of hairs. Numbers in the photograph illustrates the method used to choose the areas for measuring and comparing the hair color density.

FIG. 8B is a graph showing the quantitative analysis of the hair color density of mice given reverse osmotic water (RO) or PMT extract (1 g/kg) by oral gavage. The hair color density of mice given PMT extract was significantly higher than mice given regular water, p<0.05.

FIG. 9 is a photograph showing the comparison of the color of dorsal newly grown hairs among mice given reverse osmotic water (H₂O), mice given water containing 0.1 mg/ml PMT extract (ad libitum), and mice given PMT extract (1 mg/mL) by oral gavage (oral).

FIG. 10A is a chromatogram of 2,3,5,4′-tetrahydroxystilbene-2-O-beta-D-glucoside (THSG) in methanol obtained by HPLC.

FIG. 10B is chromatogram of PMT extract in methanol obtained by HPLC.

FIG. 11 is a photograph of agarose gel electrophoresis showing mRNA level changes of melanin related genes in response to PMT extract, THSG, and naringenin. B16-F10 cells were treated with PMT-1 (10 μg/mL), THSG (10 μg/mL) and naringenin (100 μM) for 3 days. RT-PCR was performed on treated cells and mRNA levels of Tyrosinase and its related proteins (TRP1, TRP2, p53 and p21) were evaluated. The mRNA levels of the housekeeping GAPDH was also measured. “C” represents cells treated with control medium (DMEM); “−” represents the negative control.

### DETAILED DESCRIPTION OF THE APPLICATION

Unless otherwise defined, all technical terms used herein have the same meaning as commonly understood by one of ordinary skill in the art to which this application belongs.

As used herein, the terms “subject,” “patient” and “individual” are used interchangeably herein, and mean a mammalian (e.g., human) subject to be treated and/or to obtain a biological sample from.

As used herein, the term “sample” is used herein in its broadest sense. For example, a sample including polynucleotides, peptides, antibodies and the like may include a bodily fluid, a soluble fraction of a cell preparation or media in which cells were grown, genomic DNA, RNA or cDNA, a cell, a tissue, skin, hair and the like. Examples of samples include biopsy specimens, serum, blood, urine, plasma and saliva.

As used herein, the term “effective amount” refers to the quantity of a component which is sufficient to yield a desired therapeutic response without undue adverse side effects (such as toxicity, irritation, or allergic response) commensurate with a reasonable benefit/risk ratio when used as described herein.

The specific effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, the term “treatment” is defined as the application or administration of a therapeutic agent to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease. For example, “treatment” of a patient in whom no symptoms or clinically relevant manifestations of a disease or disorder have been identified is preventive or prophylactic therapy, whereas clinical, curative, or palliative “treatment” of a patient in whom symptoms or clinically relevant manifestations of a disease or disorder have been identified generally does not constitute preventive or prophylactic therapy.

Methods involving conventional molecular biology techniques are described herein. Such techniques are generally known in the art.

Compositions and methods similar or equivalent to those described herein can be used in the practice or testing of the present application. Suitable compositions and methods are described below.

Polygonum multiflorum Thunb (PMT) is scandent perennial herb with thick rhizomes. It is regarded as a useful and safe nourishing drug in traditional Chinese medicine. According to the record of Ben Cao Gang Mu, it generally may have the following functions: nourishing the blood, benefiting liver and kidney, strengthening the bones and muscles, blackening the beard and hair, and treatment of seminal emission.

The present application provides novel methods for using an extract of Polygonum multiflorum Thunb (PMT) to modulate pigmentation and melanin production as well as treating diseases and conditions that may be associated with melanin deficiency.

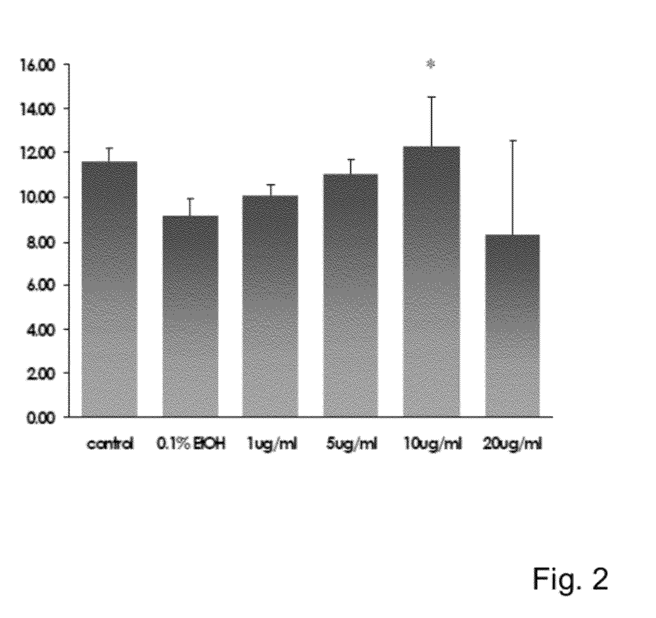

The present application demonstrated that PMT extract increased melanin production in cells, as illustrated in Example 2 (see FIG. 2).

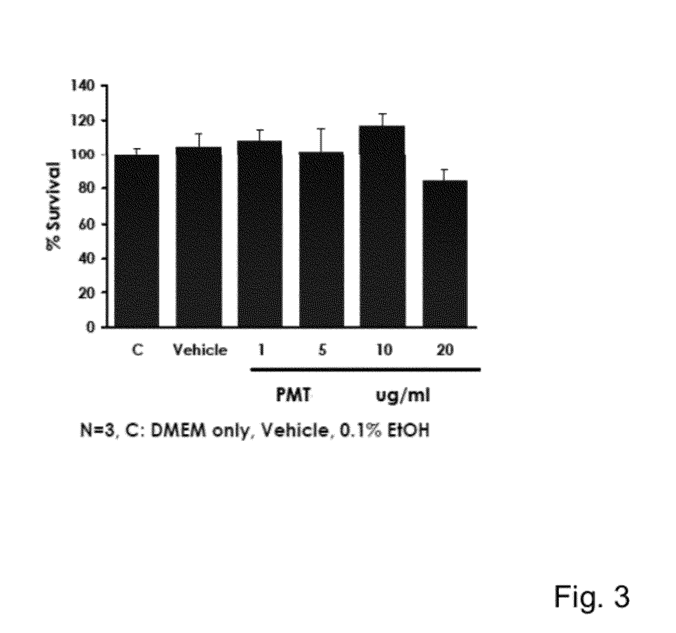

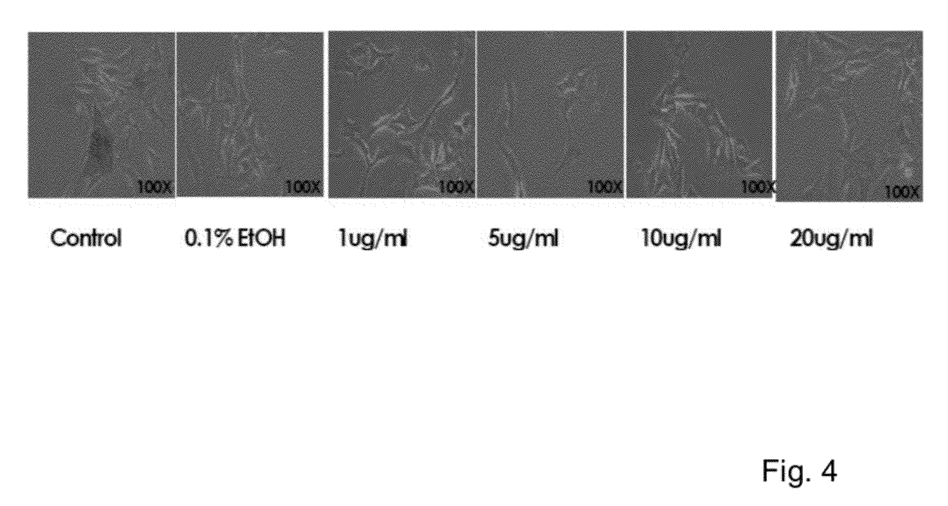

The present application further demonstrated that cells treated with PMT extract showed no signs of cytotoxicity (see FIG. 3) and no abnormal cell morphological changes, as illustrated in Example 3 (see FIG. 4).

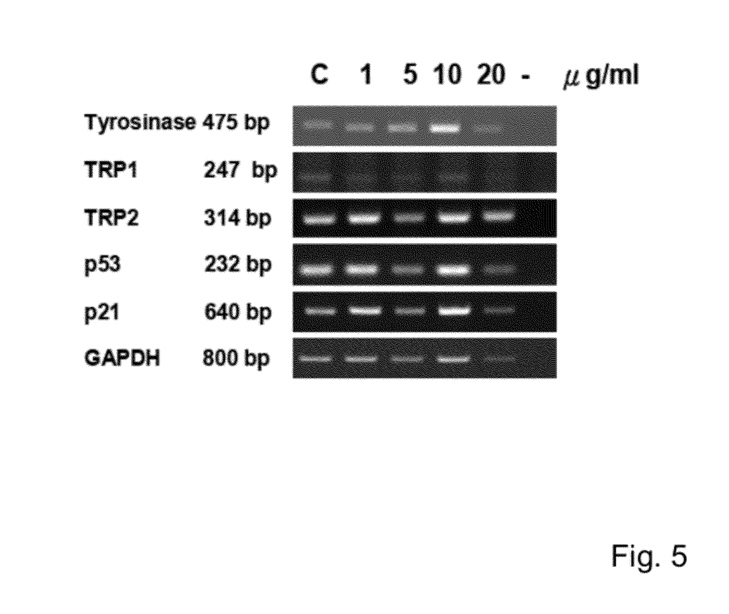

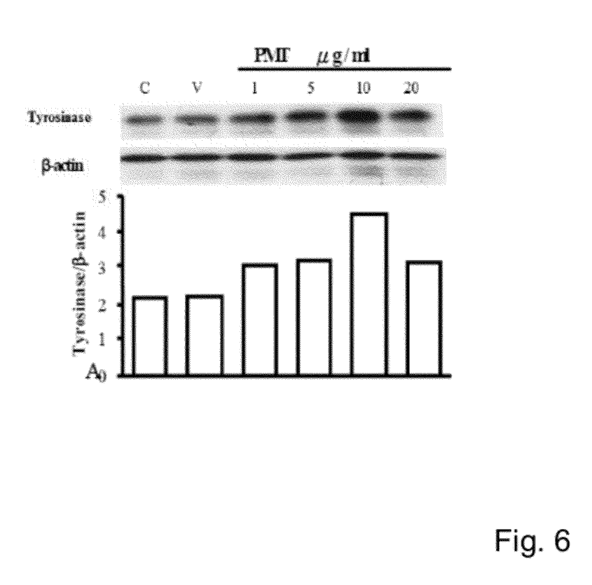

The present application demonstrated that the ability of PMT extract to increase melanin production is associated with an increase in mRNA and protein expression levels of tyrosinase, a critical enzyme required for the process of melanogenesis, as illustrated in Example 4 (see FIGS. 5 and 6).

There are two forms or types of melanin: eumelanin and pheomelanin. They are formed in melanosomes from the precursor tyrosine through a series of oxidative steps (Park et al., Cell. Mol. Life Sci. Vol. 77, pp. 1493-1506, 2009). Tyrosinase represents the critical regulatory point in the pathway of melanin formation and is the key lesion in many types of albinism (Hearing & Tsukamoto, FASEB J., Vol. 5, pp. 2902-2909, 1991). Tyrosinase mutations including missense, nonsense, frameshift and deletion mutations that lead to inactivation of the enzyme are the cause of oculocutaneous albinism, a group of hereditary disorders characterized by melanin deficiency or absence (Park et al., Cell. Mol. Life Sci. Vol. 77, pp. 1493-1506, 2009). Thus, the present application described a role for PMT extract in inducing and increasing the mRNA and protein expression levels of tyrosinase, a critical enzyme required for the process of melanogenesis, which resulted in the induction and increase in melanin production.

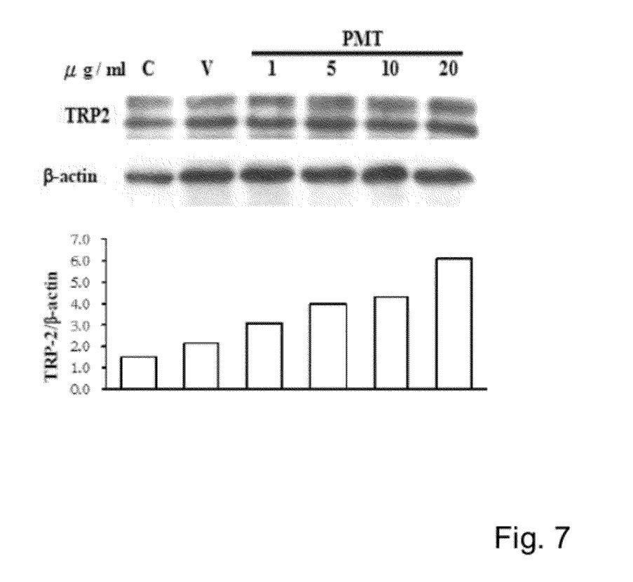

The present application demonstrated that PMT extract also increased mRNA levels of other melanogenesis associated genes, including tyrosinase-related protein 1 (TRP1) and tyrosinase-related protein 2 (TRP2), as illustrated in Example 4 (see FIG. 5). TRP-1 and TRP-2 are structurally related to tyrosinase and share about 40% amino acid homology, suggesting that they originated from a common ancestral gene. TRP-1 and TRP-2 reside within the melanosomes and, like tyrosinase, span the melanosomal membrane, although their precise function is not well elucidated (Park et al., Cell. Mol. Life Sci. Vol. 77, pp. 1493-1506, 2009). An increase in protein expression levels of TRP2 was also observed in cells treated with PMT extract, as illustrated in Example 4 (see FIG. 7).

The present application further demonstrated that PMT extract also increased the mRNA levels of genes, including the tumor suppressor gene p53 and p21, as illustrated in Example 4 (see FIG. 5). The tumor suppressor protein p53, when activated, upregulates the level of tyrosinase mRNA and protein, enhancing melanogenesis (Park et al., Cell. Mol. Life Sci. Vol. 77, pp. 1493-1506, 2009). It was reported that the p21 molecule acts as a downstream effector of p53 by activating cell cycle arrest and that p53 and p21 play a role in the human skin response to UV exposure (Ponten et al., J. Invest. Dermatol., Vol. 105, pp. 402-406, 1995).

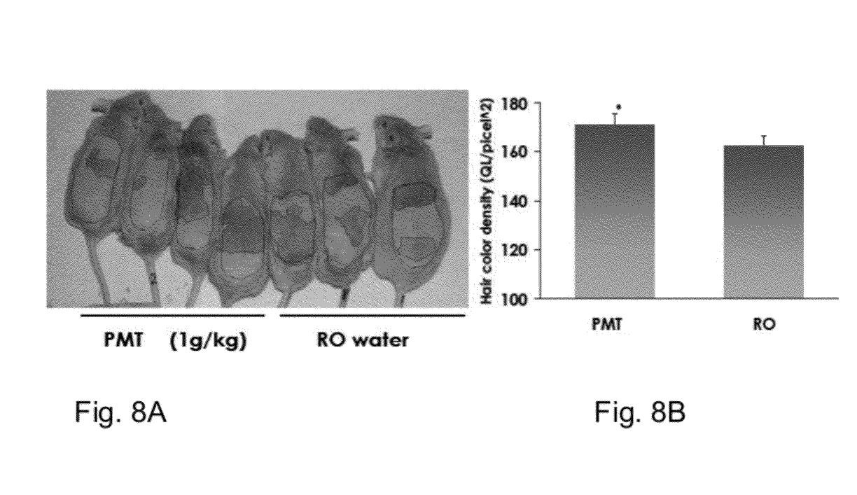

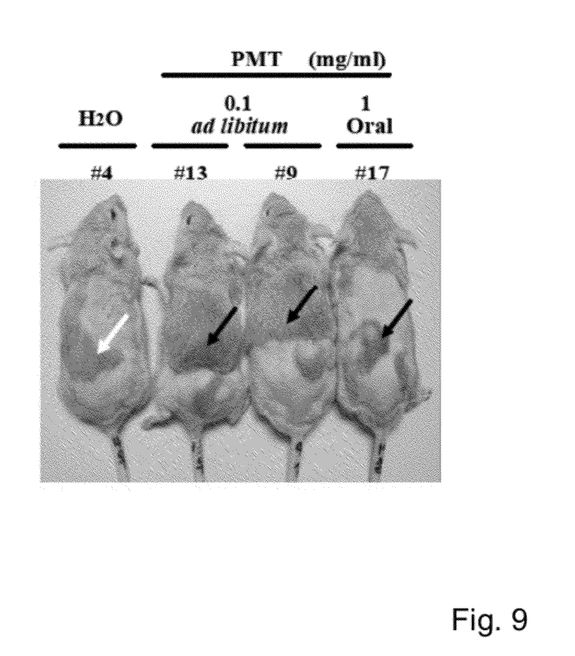

The present application demonstrated that the increase in melanin production was also observed in animals (mice) administered with PMT extract and resulted in statistically darker hair color density as compared to control animals, as illustrated in Example 5 (see FIGS. 8A, 8B and 9).

Accordingly, in one embodiment, the present application provides a method for modulating pigmentation of hair, skin, nail and/or eyelashes comprising administering to a subject in need thereof an effective amount of an extract of Polygonum multiflorum Thunb (PMT). In some embodiments, the modulation increases pigmentation of the hair, skin, nail and/or eyelashes. In some embodiments, the modulation increases pigmentation of the hair. In some embodiments, the extract of Polygonum multiflorum Thunb (PMT) is effective in increasing tyrosinase expression and/or activity. In some embodiments, the extract of Polygonum multiflorum Thunb (PMT) is effective in increasing tyrosinase-related protein 2 expression and/or activity.

In another embodiment, the present application relates to a method for modulating melanin production comprising administering to a subject in need thereof an effective amount of an extract of Polygonum multiflorum Thunb (PMT). In some embodiments, the modulation increases melanin production. In some embodiments, the extract of Polygonum multiflorum Thunb (PMT) is effective in increasing tyrosinase expression and/or activity. In some embodiments, the extract of Polygonum multiflorum Thunb (PMT) is effective in increasing tyrosinase-related protein 2 expression and/or activity.

In yet another embodiment, the present application relates to a method for treating a disease which exhibits a melanin deficiency comprising administering to a subject in need thereof an effective amount of an extract of Polygonum multiflorum Thunb (PMT). In some embodiments, the extract of Polygonum multiflorum Thunb (PMT) is effective in increasing tyrosinase expression and/or activity. In some embodiments, the extract of Polygonum multiflorum Thunb (PMT) is effective in increasing tyrosinase-related protein 2 expression and/or activity.

Conditions associated with melanin abnormalities include, but are not limited to, albinism, vitiligo, tinea versicolor, injury or inflammation, acanthosis nigricans, aging, stress, endocrine disorder. The present application thus provides useful methods for modulating pigmentation or melanin production in subjects in need thereof comprising administering an extract of PMT.

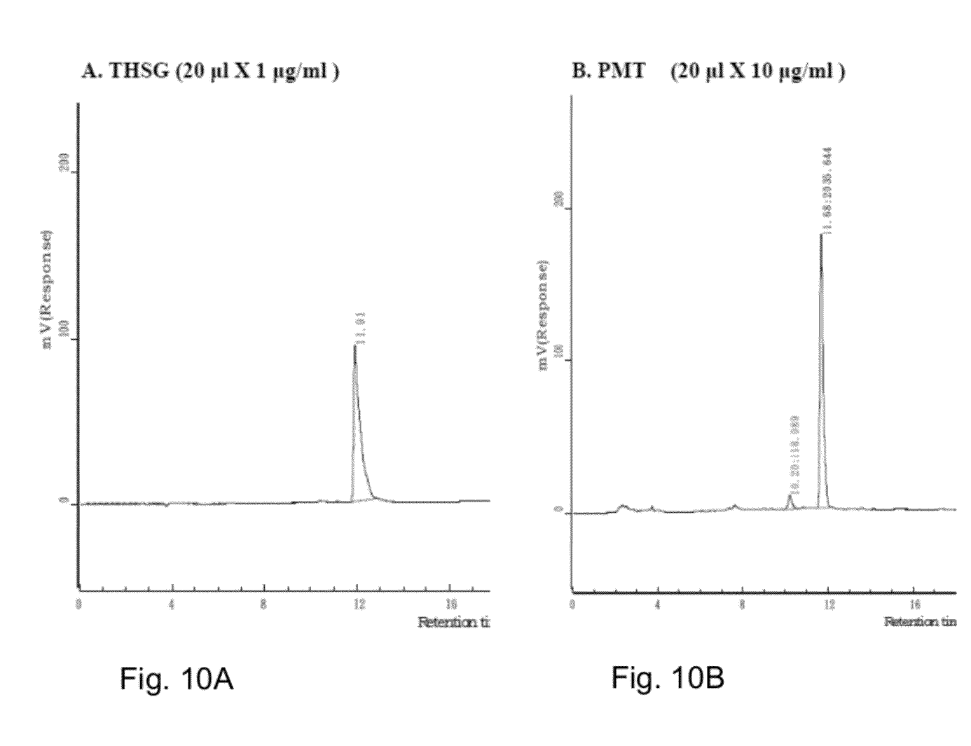

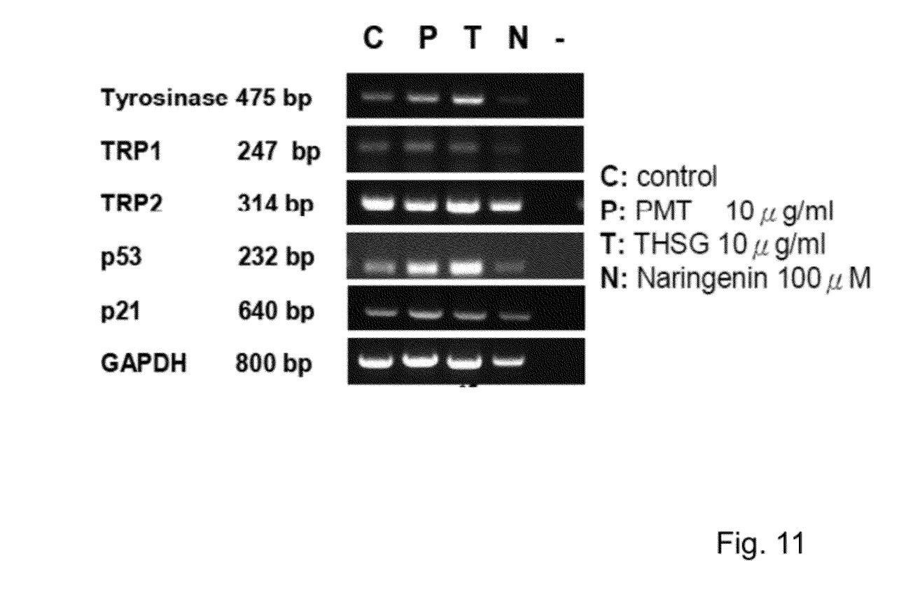

The present application further demonstrated that the effects of PMT extract may be associated with the 2,3,5,4′-tetrahydroxystilbene-2-O-beta-D-glucoside (THSG) component within the extract of PMT. As illustrated in Example 6, HPLC analysis revealed that the peak of THSG overlaps with the peak of PMT extract (see FIG. 10). This suggested that the activities of PMT extract may be attributable to THSG. In order to confirm this, the present application demonstrated that THSG was also able to increase the mRNA levels of tyrosinase as illustrated in Example 6 (see FIG. 11). THSG was also able to increase the mRNA levels of TRP1 and TRP2, as well as p53 and p21, as illustrated in Example 6 (see FIG. 11). These results suggest that THSG may be one of the active components within PMT extract that are involved in the induction of melanogenesis related genes and melanin production.

Accordingly, in some embodiments, the methods of the present application comprise PMT extract comprising 2,3,5,4′-tetrahydroxystilbene-2-O-beta-D-glucoside (THSG).

The subjects of the present application may include mammals, including but not limited to, rodent, humans, sheep, and rabbit, dog, cat. In some embodiments, the subject is a human.

The PMT extract of the present application may be administered by any means, including but not limited to, orally, topically, intravenously, intraperitoneally, subcutaneously, intramuscularly, intrathecally, intradermally, nasally, enterically, pessaries, suppositories. In some embodiments, the PMT extract is administered orally or topically.

The PMT extract of the present application may be contained in any appropriate amount in any suitable carrier substance, and is generally present in an amount from about 1% to about 95% by weight of the total weight of the composition. In some embodiments, the PMT extract is present in an amount from about 1% to about 90%, about 1% to about 80%, about 1% to about 70%, about 1% to about 60%, about 1% to about 50%, about 1% to about 40%, about 1% to about 30%, about 1% to about 20%, about 1% to about 10% and about 1% to about 5%. In some embodiments, the PMT extract is present in an amount less than about 1% by weight of the total weight of the composition. In some embodiments, the PMT extract is present in amount from about 0.5% to about 1%. In some embodiments, the PMT extract is present in an amount from about 5 μg/mL to about 10 μg/mL.

Compositions as described herein may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation.

Compositions for parenteral use may be provided in unit dosage forms (e.g., in single-dose ampules), or in vials containing several doses and in which a suitable preservative may be added. The composition may be in the form of a solution, a suspension, an emulsion, an infusion device, or a delivery device for implantation, or it may be presented as a dry powder to be reconstituted with water or another suitable vehicle before use. The PMT of the present application may be preferable in solution form in certain applications. In other applications, the PMT of the present application may be preferable in other forms, such as gel, cream, ointment, drops, injection, spray, solid forms such as tablets, and the like.

Apart from the active agent, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

A viscosity-increasing agent (such as a thickener or gelling agent) might be desirable. Exemplary viscosity-increasing agents include but are not limited to carboxymethyl cellulose (CMC), hydroxypropyl methylcellulose (HPMC), methyl cellulose, methyl hydroxylethyl cellulose (MHEC), hydroxyethyl cellulose, sodium hydroxyalkyl celluloses, and admixtures thereof.

It may be necessary to adjust the pH of the composition for particular applications. For example, in applications where the PMT extract might be acidic on production, a base, typically but not exclusively sodium hydroxide solution, can be added to adjust the pH to the desired pH or to physiological pH. Alternatively, if the PMT extract is basic on production, an acid, typically but not exclusively either hydrochloric acid or acetic acid, can be added to return the pH to the desired pH or to physiological pH. In some embodiments, it might be desirable for the composition to be at some non-neutral or non-physiological pH, in which case additional adjustments would be made. In extreme cases, a far-from-neutral composition might be needed, in which case an additional buffer might be needed. Such buffers are well known to practitioners of the art and a variety is available for use.

The PMT extract composition (such as strength of ingredients) can be tailored to the specific needs of an individual. The PMT extract composition of the present application can be applied in conjunction with dressings, such as medical dressings. Preferably, the dressing material can be a non-toxic material that will release the PMT extract into the areas as desired. Appropriate dressing materials will depend upon the nature of the site and the overall condition of the subject.

Compositions as described herein may also be combined with a second or more pharmacologically active agent, for the modulation of pigmentation, melanin production and/or treatment of complications resulting from or associated with melanin abnormalities.

The PMT extract of the present application may be administered as a bolus or as multiple doses over a period of time depending on the overall condition of the patient and medical attention needed.

The PMT extract of the present application may be administered to many areas including but not limited to hair, skin, nail, eyelashes, eye, uvea, inner ear, meninges, bones, heart, nictitans, harderian gland, choroid, and retina. Preferably, the PMT extract of the present application is administered to hair, skin, nail or eyelashes. The PMT extract of the present application may also be administered to sites, tissues and/or cells where melanogenesis occurs. Such sites, tissues and/or cells include but are not limited to, for example, hair (hair follicles), skin, nail and eyelashes. The PMT extract of the present application may also be administered to sites, tissues and/or cells where melanin-producing cells, such as melanocytes, reside including but are not limited to, for example, hair, skin, nail and eyelashes.

### EXAMPLES

The present application is further illustrated by the following specific examples. The examples are provided for illustration only and should not be construed as limiting the scope of the application in any way.

### Example 1

### PMT Extraction And Cell Culture

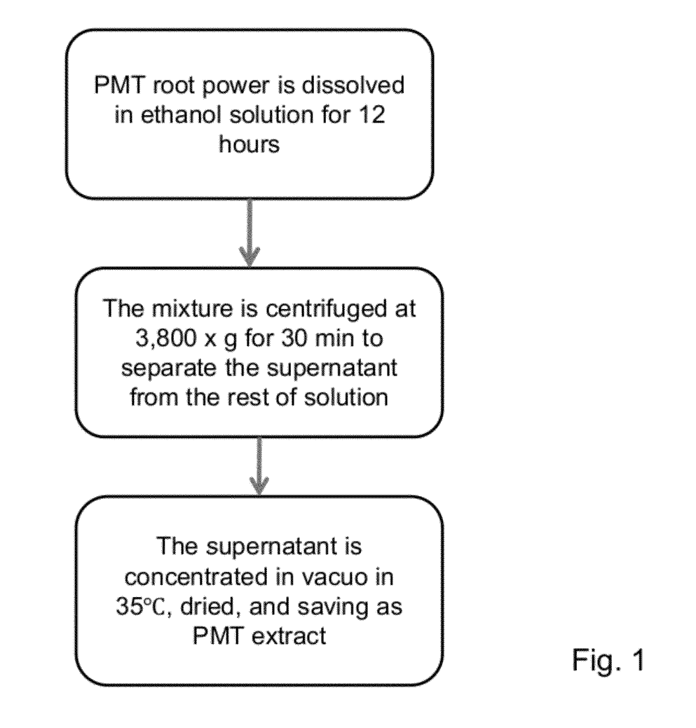

Dry Polygonum multiflorum Thunb (PMT) root powder provided by Taiwan King Herb Inc. contained the following main ingredients: gallic acid, catechin, procyanidin, emodin, galloylcatechin, gallotannin 2,3,5,4, tetahydroxystilbene-2-O, and 2,3,5,4′-tetrahydroxystilbene-2-O-beta-D-glucoside (THSG) (Yi et al., Phytochem. Anal. Vol. 18, pp. 181-187). The extraction of the root of PMT is illustrated in FIG. 1. Briefly, dry PMT root powder was mixed with ethanol at room temperature for 12 hours. The mixture was centrifuged at 3,800×g for 30 min and the pellet were removed. The supernatant was then dried by a lyophilizer and stored as a PMT extract.

The cells tested in the present application were B16-F10 mouse melanoma cells purchased by the Bioresource Collection and Research Center (BCRC). B16-F10 cells were maintained in Dulbecco's modified Eagle's medium (DMEM), 4 mM L-glutamine adjusted, 1.5 g/L sodium bicarbonate, 4.5 g/L glucose, and 10% fetal bovine serum. All cells were maintained in a humidified 37° C., 5% (v/v) CO₂ incubator.

### Example 2

### PMT Root Extract Increases Melanin Content in B16-F10 Cells

A cell suspension containing 10⁵ B16-F10 cells were placed per well into 6-well plates. Plates were incubated in a humidified 37° C., 5% CO₂ incubator. Different concentrations of PMT extract (1, 5, 10 and 20 μg/mL) were added to the cell cultures. After three days, cells were trypsinized from plates and cell suspensions were transferred to sterile centrifuge tubes. Cell suspensions were centrifuged at 2500 rpm for 5 minutes. After sitting at room temperature for 60 minutes, cell suspensions were mixed with DMSO containing 1% NaOH and the tubes were placed in a 80° C. water bath for 60 minutes. After cooling to room temperature, the melanin content was measured at 475 nm using a spectrophotometer. As illustrated in FIG. 2, PMT at a final concentration of 10 μg/mL increased melanin production in B16-F10 cells.

### Example 3

### Cell Viability in Medium Containing PMT

Surviving cell numbers were determined indirectly using the 3-(4,5-dimethylthiazol-2-yl)-2,5-diphenyltetrazolium bromide (MTT) dye reduction assay. MTT is a yellow, water-soluble tetrazolium dye that is reduced by live cells to a purple formazan product that is insoluble in aqueous solutions. The amount of MTT-formazan produced can be determined spectrophotometrically once solubilized in a suitable solvent.

A cell suspension containing 10⁴ of B16-F10 cells was placed per well into 96-well plates and cultured until cells reached 50% confluence. Cells were treated for 48 hours with PMT extract at different concentrations (1, 5, 10 and 20 μg/mL).

After the 48 hour period, 20 μL of MTT (5.0 mg/ml in PBS) was added to all wells and incubated at room temperature for 2-4 hours in a dark room. The medium was then removed and the cells were mixed with 100 μL DMSO and incubated at room temperature for 30 min. The released formazan was read at wavelengths 570 nm and 630 nm, and cell survival rates were measured for all wells. Cell morphological changes were also determined using microscopy (at 100× magnification).

Compared to the control, B16-F10 cells treated with different concentrations of PMT extract displayed no signs of cytotoxicity (see FIG. 3) and no abnormal cell morphological changes (see FIG. 4). “C” represents cells treated with control medium (Dulbecco's modified Eagle's medium (DMEM)); and “V” represents cells treated with vehicle medium (DMEM containing 0.1% ethanol (EtOH)).

### Example 4

### Analysis of Gene Expression of Melanogenesis Related Proteins in Response to PMT Root Extract

A. Reverse Transcription-Polymerase Chain Reaction (RT-PCR)

mRNA isolation: Cells were cultured in medium containing PMT extract (at 1, 5, 10 and 20 μg/mL) for 48 hours. Cells were then homogenized in TRIZOL reagent (1 mL per 10 cm² of the culture dish) and allowed to incubate for 5 minutes at room temperature. After centrifugation to remove the cell debris, the supernatant was transferred to a new tube. Approximately 0.2 μL of chloroform per 1 mL of TRIZOL reagent was added to the cells, which were then vortexed vigorously for 15 seconds and incubated at room temperature for 2-3 min. Cells were then centrifuged at no more than 12,000×g for 15 minutes at 2 to 8° C. The upper aqueous phase was carefully transferred without disturbing the interphase into fresh tubes. Approximately 0.5 mL of isopropyl alcohol per 1 mL of TRIZOL reagent used for the initial homogenization was added to the aqueous phase. The solution was incubated at 15 to 30° C. for 10 minutes and centrifuged at not more than 12,000×g for 10 minutes at 2 to 4° C. After removing the supernatant completely, the RNA pellet was obtained. The pellet was further washed once with 75% alcohol and twice with DEPC-treated water. The concentration of RNA was measured by spectrophotometric analysis and agarose gel electrophoresis.

Reverse transcription (RT): 22 μg of isolated RNA was mixed with 20 U DNAse I at 37° C. for 30 minutes. Phenol/chloroform at pH 4.0 was further added to remove any contaminating DNA. Approximately 2 μg of the RNA was first incubated at 70° C. for 10 minutes in a PCR machine. This was followed by the addition of 13.5 μL of a mixture solution containing 4 μL of 5× RT Buffer, 2 μL of 10 mM dNTP, 0.5 μg (oligo dT)18 primer, 20 U of RNase inhibitor, and double distilled water to the RNA and incubation for another 2 minutes. The RT reaction was then extended at 42° C. for 65 minutes upon the addition of 1.5 μL reverse transcriptase (300 U). The RT reaction was deactivated by incubating the reaction at 72° C. for 10 minutes. When the temperature was decreased to 4° C., the RT reaction was completed. The RT sample was stored at −20° C.

Polymerization Chain Reaction (PCR): Approximately 1 μL of sample was mixed with a solution containing 2 μL 10× PCR buffer, 2 mM of dNTP, 1 μL of corresponding primers (see Table 1 below), and 1 μL of Taq DNA polymerase. For the control group, 1 μL of water instead of sample was mixed with the solution. Initial denaturation was at 95° C. for 5 minutes. The PCR amplification reaction was adjusted in accordance to individual genes. Taking tyrosinase as an example, the following amplification reaction was used: 28 to 30 cycles of 95° C. for 45 seconds (denaturing step), 50° C. for 55 seconds (annealing step), followed by 72° C. for 1 minute (extension step). Following the 28 to 30 cycles, the PCR reaction was incubated at 72° C. for 10 minutes, and then held at 4° C. The PCR reaction protocols for the other genes: TRP1, TRP2, p53, p21 and GAPDH are the same as for tyrosinase except for the annealing temperatures and the numbers of cycles, which are provided in Table 2.

TABLE 1

| Gene | Primer Pairs | Size |
|---|---|---|
| mTyr | 5′-GGCCAGCTTTCAGGCAGAGGT-3′ | 475 bp |
| (Tyrosinase) | 5′-TGGTGCTTCATGGGCAAAATC-3′ | |
| mTrp1 | 5′-GCTGCAGGAGCCTTCTTTCTC-3′ | 247 bp |
| (Tyrosinase-Related | 5′-AAGACGCTGCACTGCTGGTCT-3′ | |
| Protein1) | | |
| mTrp2 | 5′-CCTGGCCAAGAAGAGTATCC-3′ | 314 bp |
| (Tyrosinase-Related Protein | 5′-CACGTCACACTCGTTCTTCC-3′ | |
| 2) | | |
| mp53 | 5′-AGAGACCGCCGTACAGAAGA-3′ | 232 bp |
| | 5′-CTGTAGCATGGGCATCCTTT-3′ | |
| mp21 | 5′-GTCAGAGTCTAGGGGAATTG-3′ | 640 bp |
| | 5′-TAAGACACACAGAGTGAGGG-3′ | |
| mGAPDH | 5′-CGTCCCGTAGACAAAATGGT-3′ | 800 bp |
| (Glyceraldehyde 3- | 5′-TGCTTCACCACCTTCTTGAT-3′ | |
| phosphate dehydrogenase) | | |

TABLE 2

| Gene | Annealing Temperature | Cycles |
|---|---|---|
| Trp1 | 52° C. | 28 |
| Trp2 | 52° C. | 28 |
| p53 | 50° C. | 25 |
| p21 | 51° C. | 30 |
| GAPDH | 55° C. | 22 |

The PCR reaction products were analyzed by agarose gel electrophoresis. As displayed in FIG. 5, PMT extract increased the mRNA levels of tyrosinase in B16-F10 cells, with the highest level observed at a concentration of 10 μg/mL. FIG. 5 also illustrated that PMT extract at a concentration of 10 μg/mL increased the mRNA levels of tyrosinase-related protein 1 (TRP1) and tyrosinase-related protein 2 (TRP2). FIG. 5 also demonstrated that PMT extract increased the mRNA levels of the tumor suppressor gene p53 and its downstream gene p21. The gene expression levels were normalized to the expression levels of glyceraldehyde 3-phosphate dehydrogenase (GAPDH), a housekeeping gene.

B. Western Blot Analysis

Medium containing different final concentrations of PMT extract (1, 5, 10 and 20 μg/mL) were added to cell cultures that had achieved 50% confluence. Cells were removed from culture dishes by trypsin/EDTA and transferred to a centrifuge tube after reaching 80% to 100% confluence. Cells were centrifuged at 300×g for 5-7 minutes. After the supernatant was removed, the cell pellet was resuspended in homogenate for final concentration of 250 mM sucrose based lysis buffer containing 1 mM EDTA, 1 mM PMSF, 10 mM Tris-HCl, vortexed vigorously, and incubated at 4° C. for 30 minutes. The cell pellet was centrifuged at 12,750×g at 4° C. for 20 minutes. The supernatant was transferred to a new tube and the protein concentration was further measured by Bradford assay.

SDS-PAGE gel electrophoresis under denaturing conditions was performed. 10 μg of the isolated proteins were loaded per well and ran on an SDS-PAGE gel containing a 5% (w/v) stacking gel and a 8% (w/v) separating gel.

Immunoblotting to a 0.45-μm polyvinylidene fluoride (PVDF) membrane in a tank transfer system was performed. The PVDF was washed with PBST and blocking buffer containing 5% (w/v) nonfat dry milk was added to the PVDF to block nonspecific binding sites. The PVDF was incubated in the blocking solution on a shaker for 2 hours; then the PVDF was washed with PBST.

The PVDF was incubated in PBS containing appropriate concentrations of primary antibodies and incubated at 4° C. for 12-24 hours. The primary antibodies used include antibodies directed to mouse tyrosinase (Santa Cruz Biotechnology, Inc., at 1:2000 dilution), mouse TRP1 (tyrosinase-related protein 1) (Santa Cruz Biotechnology, Inc., at 1:2000 dilution), mouse TRP2 (tyrosinase-related protein 2) (Santa Cruz Biotechnology, Inc., at 1:2000 dilution) and mouse β-actin (Novus Biologicals, Inc., at 1:10000 dilution). The PVDF was then washed with PBST for 20 minutes. The PVDF was then incubated with PBS containing appropriate concentrations of secondary antibodies and placed on a shaker at room temperature. The secondary HRP-conjugated antibodies used include donkey anti-goat IG-HRP (Santa Cruz Biotechnology, Inc., at 1:10000 dilution) and goat anti-mouse IgG (Invitrogen Corporation, at 1:20000 dilution). Finally, the PVDF was washed with PBST for 20 min.

Detection was carried out using the ECL detection system. Specifically, the PVDF membrane was incubated with ECL detection solution for 1 min at room temperature. After draining off excess detection solution, the PVDF was wrapped in plastic wrap and immediately exposed to film. The film was then analyzed using the Multi Gauge V 3.0.

As illustrated in FIG. 6, PMT extract increased the tyrosinase protein expression in B16-F10 cells and that the highest expression occurred using a final PMT extract concentration of 10 μg/ml.

FIG. 7 illustrates that PMT extract increased the TRP-2 (tyrosinase-related protein 2) expression in B16-F10 cells and that the highest expression occurred using a final PMT extract concentration of 20 μg/mL.

### Example 5

### PMT Increases Pigmentation of Mouse Hair

Transgenic mice were prepared by random integration of a DNA fragment encoding mouse tyrosinase injected into a fertilized (0.5 day before microinjection) egg's pronucleus of the albino mice. The transgenic mice were produced as described (Hsiao et al., Genesis, Vol. 39, pp. 122-129, 2004).

The mice were kept on a 12:12 hour artificial light-dark regimen in a specific pathogen free (SPF) area at 22° C.±2° C. and 50%±20% relative humidity. The mice had free access to food and water.

Mice were fed for two months with regular water (RO water) or water containing different concentrations of PMT extract. Mice were given PMT extract either through oral gavage or free access (ad libitum). On the first day of feeding, the mice dorsal hair was shaved and photographs of the dorsal areas were taken every two weeks. The photos were analyzed by FUJIFIM Multi Gauge V 3.0 to measure the color changes in those areas.

Statistical analysis consisted ANOVA and Duncan test carried out by SPSS computer software. For all statistical analyses, a P value of less than 0.05 was considered to indicate a significant difference.

FIG. 8A illustrates the method used to choose the areas for measuring and comparing the hair color density in mice given regular water (RO water) and mice given PMT extract (1 g/kg) by oral gavage. Briefly, the area was determined by free hand drawing outline of newly grown hair and the Quantum Level (QL) value of Region of Interest (ROI) was normalized to average upper and lower background. The quantitative analysis is illustrated in FIG. 8B which shows that the hair color density of tyrosinase-expressing albino mice and given PMT extract (1 g/kg) by oral gavage was statistically significantly darker than tyrosinase-expressing albino mice fed with regular water (RO). FIG. 9 illustrates a comparison of dorsal hair color between mice given regular water (H20), mice given water containing 0.1 mg/ml PMT extract (ad libitum), and mice given PMT extract (1 g/kg) by oral gavage (oral). Similar experiments were also performed to evaluate the effect on the hairs on the ventral sides of the mice. No differences or effects were observed for the hair on the ventral sides of the mice, whether PMT extract was given or not. In contrast, hair color darkening was observed in mice given PMT extract (either by ad libitum or by oral gavage) (as indicated by the arrows). These results suggested that the effects of PMT extract were mediated via its effects on tyrosinase, resulting in a darkening of hair color of the transgenic mice.

### Example 6

### THSG Increases Melanogenesis Related Gene

High-performance liquid chromatography (HPLC) analysis of the PMT extract was conducted under the following conditions: Column C-18 (581325-U SUPELCO, Ascentic); mobile phase consisted of 80% H2O +20% MeOH to 100% MeOH; flow rate: 1 mL/min; samples' absorbance measured at 270 nm. As shown in FIGS. 10A and 10B, the first clear peak was detected around 12 minutes after running, which peaks at a similar position as 2,3,5,4′-tetrahydroxystilbene-2-O-beta-D-glucoside (THSG). These results indicate that one of the active ingredients of PMT extract may include THSG.

To determine whether THSG had similar effects as the PMT extract on the induction of melanin related gene expression, B16-F10 cells were cultured in medium containing PMT extract (10 μg/mL), THSG (10 μg/mL), naringenin (100 μM) for three days and RT-PCR was performed to analyze melanin biosynthesis related gene expression using the protocol as described in Example 4 above. As illustrated in FIG. 11, both PMT extract and THSG at 10 μg/ml of the final concentration increased the mRNA level of tyrosinase in B16-F10 cells. FIG. 11 also shows that PMT extract and THSG at 10 μg/ml of the final concentration increased the mRNA level of tyrosinase-related protein 1 (TRP1) and tyrosinase-related protein 2 (TRP2). FIG. 11 further shows that PMT extract and THSG increased the mRNA level of tumor suppressor protein p53 and its downstream gene p21. However, as shown in FIG. 11, there is no increase of the mRNA level of melanogenesis related genes in response to naringenin at 100 μM of the final concentration. These results suggest that THSG may be one of the active agents within PMT extract, as evidenced by its ability to increase and enhance gene expression of melanogenesis-related genes.

All references, including publications, patent applications and patents, cited herein are hereby incorporated by reference to the same extent as if each reference was individually and specifically indicated to be incorporated by reference and was set forth in its entirety herein.

Any combination of the above-described elements in all possible variations thereof is encompassed by the application unless otherwise indicated herein or otherwise clearly contradicted by context.

The terms “a” and “an” and “the” and similar referents as used in the context of describing the application are to be construed to cover both the singular and the plural, unless otherwise indicated herein or clearly contradicted by context.

The use of any and all examples, or exemplary language (e.g., “such as”) provided herein, is intended merely to better illuminate the application and does not pose a limitation on the scope of the application unless otherwise indicated. No language in the specification should be construed as indicating any element is essential to the practice of the application unless as much is explicitly stated.

The description herein of any aspect or embodiment of the application using terms such as “comprising,” “having,” “including” or “containing” with reference to an element or elements is intended to provide support for a similar aspect or embodiment of the application that “consists of,” “consists essentially of,” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context). That said, the terms “comprising,” “having,” “including” or “containing” in the claims should be construed according to the conventional “open” meaning of those terms in the patent law to include those elements enumerated as well as other elements. Likewise, the terms “consisting of,” “consists of,” “consists essentially of,” or “substantially comprises” should be construed according to the “closed” or “partially closed” meanings ascribed to those terms in the patent law.

This application includes all modifications and equivalents of the subject matter recited in the aspects or embodiments presented herein to the maximum extent permitted by applicable law.

What is claimed is:

1. A method for modulating pigmentation of hair, skin, nail and/or eyelashes comprising administering to a subject in need thereof an effective amount of an extract of Polygonum multiflorum Thunb (PMT).

2. A method for modulating melanin production comprising administering to a subject in need thereof an effective amount of an extract of Polygonum multiflorum Thunb (PMT).

3. A method for treating a disease which exhibits a melanin deficiency comprising administering to a subject in need thereof an effective amount of an extract of Polygonum multiflorum Thunb (PMT).

4. The method of claim 1, wherein said modulation increases pigmentation of said hair, skin, nail and/or eyelashes.

5. The method of claim 2, wherein said modulation increases melanin production.

6. The method of any one of claims 1-3, wherein said extract of Polygonum multiflorum Thunb (PMT) is effective in increasing tyrosinase expression and/or activity.

7. The method of any one of claims 1-3, wherein said extract of Polygonum multiflorum Thunb (PMT) is effective in increasing tyrosinase-related protein 2 expression and/or activity.

8. The method of any one of claims 1-3, wherein said subject is a human.

9. The method of any one of claims 1-3, wherein said extract of PMT is administered orally, topically, intravenously, intraperitoneally, subcutaneously, intramuscularly, intrathecally, intradermally, nasally, enterically, pessaries, suppositories.

10. The method of claim 9, wherein said extract of PMT is administered orally and topically.

11. The method according to any one of claims 1-3, wherein said extract of PMT comprises 2,3,5,4′-tetrahydroxystilbene-2-O-beta-D-glucoside (THSG).


SEQUENCE LISTING (12 sequences)

SEQ ID NO: 1 - DNA, 21 bp, Artificial Sequence
  Primer
ggccagctttcaggcagaggt

SEQ ID NO: 2 - DNA, 21 bp, Artificial Sequence
  Primer
tggtgcttcatgggcaaaatc

SEQ ID NO: 3 - DNA, 21 bp, Artificial Sequence
  Primer
gctgcaggagccttctttctc

SEQ ID NO: 4 - DNA, 21 bp, Artificial Sequence
  Primer
aagacgctgcactgctggtct

SEQ ID NO: 5 - DNA, 20 bp, Artificial Sequence
  Primer
cctggccaagaagagtatcc

SEQ ID NO: 6 - DNA, 20 bp, Artificial Sequence
  Primer
cacgtcacactcgttcttcc

SEQ ID NO: 7 - DNA, 20 bp, Artificial Sequence
  Primer
agagaccgccgtacagaaga

SEQ ID NO: 8 - DNA, 20 bp, Artificial Sequence
  Primer
ctgtagcatgggcatccttt

SEQ ID NO: 9 - DNA, 20 bp, Artificial Sequence
  Primer
gtcagagtctaggggaattg

SEQ ID NO: 10 - DNA, 20 bp, Artificial Sequence
  Primer
taagacacacagagtgaggg

SEQ ID NO: 11 - DNA, 20 bp, Artificial Sequence
  Primer
cgtcccgtagacaaaatggt

SEQ ID NO: 12 - DNA, 20 bp, Artificial Sequence
  Primer
tgcttcaccaccttcttgat